(19) United States
(12) Patent Application Publication
(10) Pub. No.: US 2002/0116025 A1
(43) Pub. Date: Aug. 22, 2002

(54) Instrument for simulating the effect of suburethro-cervical support on urinary incontinence

(75) Inventor: Francois Haab, Paris (FR)

Correspondence Address: STEPTOE & JOHNSON LLP, 1330 Connecticut Avenue, N.W., Washington, DC, 20036, US

(73) Assignee: Johnson & Johnson International

(21) Appl. No.: 10/012,045

(22) Filed: Dec. 11, 2001

(30) Foreign Application Priority Data
Dec. 12, 2000 (FR) FR00 16136

(51) Int. Cl. A61B 17/28

(52) U.S. Cl. 606/206; 600/201; 606/1

(57) ABSTRACT

The present invention provides an instrument for simulating support offered to the urethra in preparation for surgery to counter urinary incontinence, the instrument comprising:

a support tool having two branches; and

a strip of controlled flexibility fixed via its respective ends to the two branches.

The present invention relates to the field of treating urinary incontinence.

### BACKGROUND OF THE INVENTION

As shown diagrammatically in FIG. 1, in women the urethra U is placed immediately above the wall of the vaginal V and is supported by muscle, ligaments, and conjunctive tissue M. This support system contributes actively to the stress continence mechanism. Thus, stress urinary incontinence in women is usually associated with slackening of these urethro-cervical support structures. The most usual technique for treating urinary incontinence in women consists in implanting a support tape around a portion of the urethra or the neck of the bladder in order to reinforce this support floor so as to reestablish one of the main mechanisms of continence.

Reference can usefully be made to the document British Journal of Obstetrics and Gyneacology, April 1999, Vol. 106, pp. 345-350, “A three-year follow-up of tension-free vaginal tape for surgical treatment of female stress urinary incontinence”, by Ulf Ulmsten, Per Johnson, and Masoumeh Rezapour, in order to obtain a good understanding of that known technique for treating incontinence by implanting tapes.

Those known means have already given good service.

Nevertheless, although rare, it can happen that implanting a support tape does not always stop incontinence, particularly when the physiopathological mechanism involved associates suburethral slackening and sphincter insufficiency.

In this respect, proposals have indeed been made to test the effect of reinforcing the support floor offered to the urethra, prior to surgery.

For this purpose, the surgeon supports the urethra temporarily by placing fingers in the patient's vaginal canal and asking her simultaneously to cough so as to observe the effect on the urethra of the muscle tissue slackening due to the cough.

Nevertheless, that test method does not always give satisfaction. It is often difficult for the surgeon, particularly an inexperienced surgeon, to determine accurately the position and the strength of the artificial support presented in this way.

Furthermore, like all surgery, the prospect of implanting a tape gives rise to non-negligible apprehension with most patients concerned.

Thus, it appears that presently known means do not provide all the expected comfort.

### OBJECTS AND SUMMARY OF THE INVENTION

An object of the present invention is to improve this situation.

In the context of the present invention, this object is achieved by an instrument for simulating support offered to the urethra in preparation for surgery to counter urinary incontinence, the instrument comprising:

a support tool having two branches; and

a strip of controlled flexibility fixed via its respective ends to the two branches.

Thus, because of the invention, it suffices for the surgeon to insert the tape fixed to the two branches of the support tool into the vaginal canal of a patient, beneath the urethra, in order to simulate having the support tape implanted.

If incontinence is eliminated with such support (i.e. if there is no loss of urine when coughing is provoked), then the patient is reassured as to the result of the surgery.

On the contrary, if the incontinence persists, then the surgeon can avoid pointless and traumatizing surgery.

### BRIEF DESCRIPTION OF THE DRAWINGS

Other characteristics, objects, and advantages of the present invention will appear on reading the following detailed description and on observing the accompanying drawing, given by way of non-limiting example, and in which:

FIG. 1, described above, is a diagram showing the anatomy of the urinary organs in women;

FIG. 2 shows an instrument constituting a first embodiment of the present invention;

FIG. 3 shows an instrument constituting a second embodiment of the present invention;

FIG. 4 is a perspective side view of the second embodiment of this instrument; and

FIG. 5 shows a strip as used in preferred embodiments of the present invention.

### MORE DETAILED DESCRIPTION

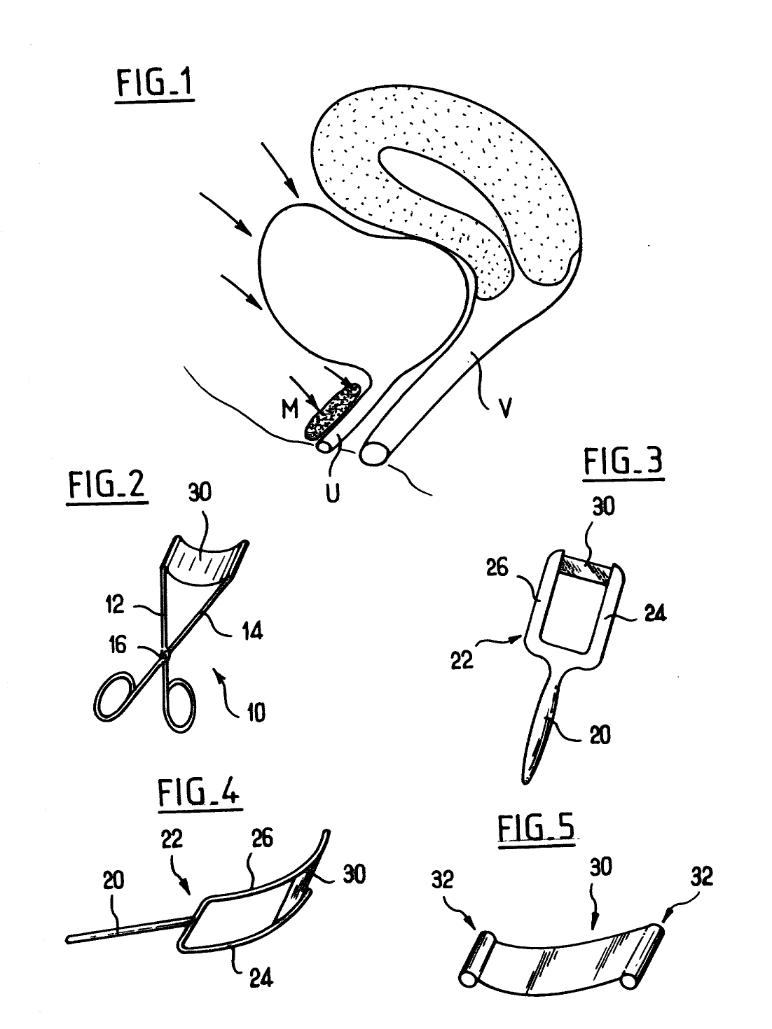

The instrument shown in FIG. 1 constitutes a first embodiment of the present invention and comprises forceps 10 having two branches 12, 14 hinged to pivot about a pin 16, and carrying a strip 30 via their respective ends.

The strip 30 is made of plastics material, preferably polypropylene, and typically it is about 1.5 centimeters (cm) wide and its length lies in the range 2 cm to 3 cm.

When the forceps 10 is in the closed position, its two branches 12 and 14 touch each other so that the instrument is relatively small. While the forceps are in this position, the strip 30 is easily inserted into the vaginal canal of a patient in order to perform the above-described test.

In contrast, when the two branches 12, 14 are spaced apart or deployed, the strip 30 is deployed beneath the urethra so as to simulate the support of the kind that is to be provided by a strip that has been implanted permanently.

In an improved embodiment, means can be provided for controlling the extent to which the two branches 12 and 14 are spaced apart. More precisely, means can be provided to define a plurality of predetermined spacings. Such means can be constituted, for example, by a crenellated connection placed between the two branches 12, 14 on their ends remote from the strip 30 and thus accessible to the practitioner.

FIG. 3 shows another embodiment of the present invention. In this embodiment, the instrument of the present invention is in the form of a tool that is generally lyre-shaped, comprising a handle 20 having one end secured to a generally U-shaped endpiece 22 having two branches 24 and 26 which carry the strip 30 at their ends.

Typically, the branches 24 and 26 are about 3 cm to 4 cm long. The spacing between the two branches 24 and 26 at their ends is typically 2 cm to 3 cm.

Accompanying FIG. 3 is a diagram of a lyre-shaped tool presenting branches 24 and 26 of fixed spacing.

In another embodiment, means can be provided enabling the spacing between the two branches 24 and 26 to be controlled and modified.

The strip 30 can be fixed permanently by any suitable means to the branches 12 & 14 or 24 & 26, e.g. by adhesive, by heat sealing, by riveting, by mechanical locking, or by any equivalent means.

In a variant, the strip 30 can be removably mounted on the ends of the branches 12 & 14 or 24 & 26.

To this end, the strip 30 can be provided at its ends with any suitable mechanical locking means complementary to arrangements provided on the ends of the branches 12 & 14 or 24 & 26.

In an embodiment that is preferred in this context, and that is shown in FIG. 5, the strip 30 is provided at each of its ends with a bushing 32 suitable for being engaged on the end of a branch 12, 14 or 24, 26 of the instrument of the invention.

Naturally, the bushings 32 could be replaced by any equivalent mechanical locking means.

In the context of the invention, it is preferable for the instrument and the strip to be for single use only.

Nevertheless, it is possible to make provision for at least the instrument to be reusable. In this context, the strip preferably has means at its ends for facilitating installation on and removal from the instrument. In particular, in this context, the strip 30 can be provided with bushings 32 or with any other equivalent mechanical locking means.

Naturally, the present invention is not limited to the particular embodiments described above, and extends to any variant in the spirit of the invention.

In particular, the present invention also provides a method of simulating the support offered to the urethra with a view to surgery to counter urinary incontinence, the method comprising the steps consisting in providing a support tool having two branches carrying a strip of controlled flexibility and fixed via respective ends to the two branches, and in inserting the tool into the vaginal canal of a patient so that the strip supports the urethra.

In the context of the invention, the method further comprises the step of observing any leakage of urine that results from provoking coughing while the urethra is being supported by the strip carried by the support tool.

The branches 12 & 14 or 26 & 24 of the instrument can be rectilinear. Nevertheless, it is preferable for the branches 12 & 14 and 26 & 24 to be curved as shown in FIGS. 2 and 4.

By way of example, in the second embodiment shown in FIGS. 3 and 4, the branches 26 & 24 can slope at an angle lying in the range 45° to 60° relative to the longitudinal axis of the handle 20.

1/ An instrument for simulating support offered to the urethra in preparation for surgery to counter urinary incontinence, the instrument comprising:
a support tool having two branches; and
a strip of controlled flexibility fixed via its respective ends to the two branches.

2/ An instrument according to claim 1, wherein the strip is made of a plastics material, e.g. polypropylene.

3/ An instrument according to claim 1, wherein the strip is about 2 cm to 3 cm long.

4/ An instrument according to claim 1, wherein the strip is about 1.5 cm wide.

5/ An instrument according to claim 1, wherein the strip is fixed in removable manner to the support tool.

6/ An instrument according to claim 1, wherein the strip has mechanical locking means at its ends that are complementary to fixing means provided on the ends of the branches of the support tool.

7/ An instrument according to claim 1, wherein the strip has bushings at its ends that are designed to be engaged on the ends of the branches of the support tool.

8/ An instrument according to claim 1, wherein the strip is permanently fixed to the ends of the branches of the support tool, e.g. by adhesive, by heat sealing, by riveting, or by mechanical locking.

9/ An instrument according to claim 1, wherein the support tool is constituted by a forceps having two pivoting branches.

10/ An instrument according to claim 9, wherein means are provided enabling the spacing of the two branches to be controlled, said means preferably being in the form of a crenellated link.

11/ An instrument according to claim 1, wherein the support tool is generally lyre-shaped comprising a handle provided with a U-shaped endpiece having two branches.

12/ An instrument according to claim 11, the instrument being provided with means enabling the spacing of the branches to be controlled.

13/ An instrument according to claim 1, wherein the two branches of the support tool are not rectilinear.

14/ An instrument according to claim 13, wherein the support tool is generally lyre-shaped comprising a handle provided with a U-shaped endpiece having two branches, and wherein the branches are inclined relative to the handle at an angle lying in the range 45° to 60°.
